(19) United States
(12) Patent Application Publication
(10) Pub. No.: US 2011/0224552 A1
(43) Pub. Date: Sep. 15, 2011

(54) ULTRASOUND ASSEMBLY AND SYSTEM COMPRISING INTERCHANGABLE TRANSDUCERS AND DISPLAYS

(75) Inventors: McKee Poland, Andover, MA (US); Martha Gail Grewe Wilson, Andover, MA (US)

(73) Assignee: KONINKLIJKE PHILIPS ELECTRONICS N.V., Eindhoven (NL)

(21) Appl. No.: 13/130,220

(22) PCT Filed: Nov. 10, 2009
(86) PCT No.: PCT/IB2009/054999

§ 371 Date: May 19, 2011

Related U.S. Application Data
(60) Provisional application No. 61/119,512, filed on Dec. 3, 2008.

(51) Int. Cl. A61B 8/00

(52) U.S. Cl. 600/459

(57) ABSTRACT

An ultrasound assembly comprises a module having an input side and an output side; an ultrasound transducer comprising a micro-beamformer configured for attachment to and detachment from the input side of the module; and a display attached to the output side of the module. An ultrasound system is also described.

### BACKGROUND AND SUMMARY

Acoustic waves (including, specifically, ultrasound waves) are useful in many scientific or technical fields, such as in medical diagnosis and medical procedures, non-destructive control of mechanical parts and underwater imaging, etc. Acoustic waves allow diagnoses and visualizations which are complementary to optical observations, because acoustic waves can travel in media that are not transparent to electromagnetic waves.

In one application, acoustic waves are employed by a medical practitioner in the course of performing a medical procedure or to provide images of a particular anatomical region of a body. Often, an acoustic imaging apparatus is employed to provide images of an area of interest to the medical practitioner to facilitate successful performance of the medical procedure.

As should be appreciated by one having ordinary skill in the art, the acoustic imaging apparatus comprises an ultrasound transducer and signal processing electronics that capture the electrical signal from the acoustic transducer and process the signal for display on one type of monitor or another. The monitor may then be viewed by the medical practitioner real-time, or may be stored/reproduced for later review, or both.

As is known, there are various types of transducers that can be used to capture ultrasonic images. For example, there are linear, curved linear and phased array transducers, which may be used in ultrasound. These transducers may have elements arranged in a one-dimensional or a two-dimensional fashion, which can enable the capturing of either a narrow slice of echo data, multiple narrow slices of echo data in different orientations with respect to each other, or a full volume set of echo data. Each type of array has advantages, and depending on the medical anatomy being imaged (due to different target depths or imaging window accessibility), a medical practitioner may select one type of transducer over another. As should be appreciated, in known systems this results in duplicative transducer electronics, transducer housings, and cables, and thus increases the overall capital expenditure for the medical facility.

Furthermore, the arrangement of the medical equipment in the imaging room can be challenging due to the placement of the ultrasound system and its display, which the user needs to look at during the scanning session. Storing and using multiple transducer probes in the imaging room exacerbates the problem of crowding the patient area with cables and equipment.

In addition, in such known systems, the main ultrasound system and its display are similarly problematic for placement, since they are typically bulky and relatively immobile. Traditional ultrasound scanners are large, weighing up to several hundred pounds, and are integrated with wheeled carts. Even newer “compact” ultrasound display systems, typically mounted semi-permanently on smaller, lighter carts, must be transported to a practical location such that the display is visible to the sonographer but the cart is sufficiently out of the way of the medical procedure. This is a difficult compromise to achieve, and often leads to awkward viewing angles or motions such as leaning across the patient by the medical practitioner to view the display.

What is needed, therefore, is an ultrasound assembly and system that overcomes at least the shortcomings of the known assemblies and systems described above.

In accordance with a representative embodiment, an ultrasound assembly comprises a module having an input side and an output side; an ultrasound transducer comprising a micro-beamformer configured for attachment and detachment from the input side of the module; and a display attached to the output side of the module.

In accordance with another representative embodiment, a system for ultrasound imaging comprises an ultrasound assembly. The ultrasound assembly comprises: a module having an input side and an output side; a ultrasound transducer comprising a micro-beamformer configured for attachment and detachment from the input side of the module; and a display attached to the output side of the module.

### BRIEF DESCRIPTION OF THE DRAWINGS

The present teachings are best understood from the following detailed description when read with the accompanying drawing figures. The features are not necessarily drawn to scale. Wherever practical, like reference numerals refer to like features.

FIG. 1 is a perspective view of an ultrasound assembly in accordance with a representative embodiment.

FIG. 2 is a simplified schematic diagram of an ultrasound assembly in accordance with a representative embodiment.

FIG. 3 is a simplified block diagram of a system for ultrasound imaging in accordance with a representative embodiment.

### DEFINED TERMINOLOGY

As used herein, the terms ‘a’ or ‘an’, as used herein are defined as one or more than one.

In addition to their ordinary meanings, the terms ‘substantial’ or ‘substantially’ mean to with acceptable limits or degree to one having ordinary skill in the art.

In addition to their ordinary meanings, the term ‘approximately’ mean to within an acceptable limit or amount to one having ordinary skill in the art. For example, ‘approximately the same’ means that one of ordinary skill in the art would consider the items being compared to be the same.

### DETAILED DESCRIPTION

In the following detailed description, for purposes of explanation and not limitation, representative embodiments disclosing specific details are set forth in order to provide a thorough understanding of the present teachings. Descriptions of known devices, materials and manufacturing methods may be omitted so as to avoid obscuring the description of the example embodiments. Nonetheless, such devices, materials and methods that are within the purview of one of ordinary skill in the art may be used in accordance with the representative embodiments.

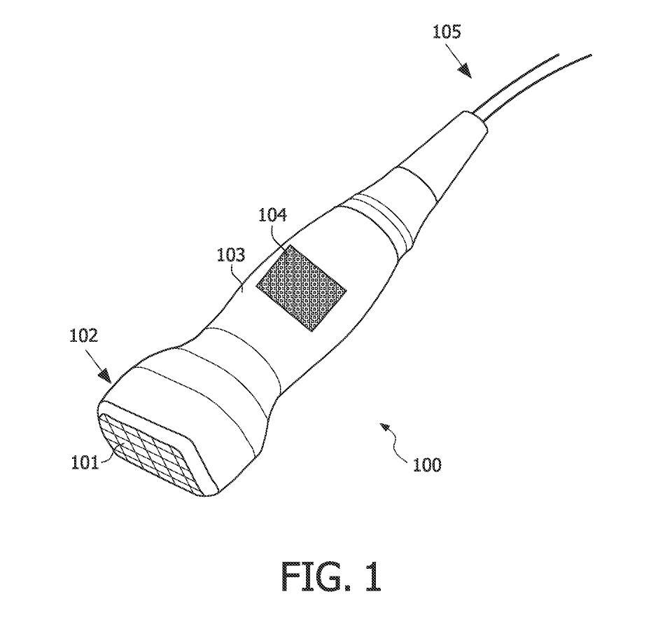

FIG. 1 is a perspective view of an ultrasound assembly 100 in accordance with a representative embodiment. The assembly comprises a phased array transducer 102 having transducer elements 101 in a forward portion thereof. The transducer elements 101 are shown in a two-dimensional array in the representative embodiment. As will become clearer as the present description continues, the transducer elements may be arranged in a linear array, or in a curved linear array, or other transducer arrangement within the purview of one having ordinary skill in the art. It is noted that a lens covering the elements 101 is normally included; but is not shown in the various Figs.

The transducer 102 is connected to an ultrasound (US) module 103 in a detachable manner. The module 103 illustratively comprises a display 104 configured to provide an ultrasound image (not shown) garnered from the transducer 102. The display 104 is illustratively a small form-factor liquid crystal display (LCD) device but may be a display based on other technologies. For example, the display 104 may be a small form-factor organic light emitting diode (OLED) device to name only one alternative to the LCD. Other types of displays based on known technologies are contemplated.

Notably, because the assembly 100 is designed and intended for hand-held use by a medical practitioner, the display 104 is beneficially of a comparatively small form-factor as mentioned above. It is contemplated that the display 104 may be the only display of an ultrasound system; or an auxiliary display used by the medical practitioner during a medical procedure or test. As should be appreciated by one having ordinary skill in the art, the locating of the display 104 fosters simplicity and accuracy during certain procedures and testing. Beneficially, because the display 104 is on the module, the medical practitioner can look to the location where he/she is physically scanning and view the resultant image on the display 104 without looking away at a remote display. For instance, the display 104 could be attached to the back of a transducer in a manner that it can be easily rotated and tilted or can be located on the side of the module 103 in a so-called “flip-out” style configuration (similar to a consumer video camera).

Moreover, the display 104 may be detachable so as to be positioned in a desired location separate from the body of the transducer and module. Among other benefits, this is useful in cases where another action is being effected simultaneously, such as placement of a needle for a biopsy, or insertion of a catheter in the body. The medical practitioner would be able to hold the assembly 100 in one hand, and guide the needle/catheter with the other using the image on the display 104 to facilitate the process and without having to look away to a remote monitor (not shown).

The module 103 may be connected to a system (not shown) via connection 105. In representative embodiments, the connection 105 may be a wireless connection configured to operate under one of a variety of wireless protocols provided under standards. Such protocols are known to one having ordinary skill in the art and thus are not detailed in order to avoid obscuring the description of the representative embodiments. Notably, however, due to issues of confidentiality related to medical information, the selected protocol will likely have a competent level of security to ensure compliance with medical information confidentiality.

Alternatively, the connection 105 is shown to be a wired connection and may be compatible with one of a variety of standards. Illustratively, the connection may be a single differential serial pair such as universal serial bus (USB) or low-voltage differential signaling (LVDS). However, it is contemplated that other types of connections may be used.

As alluded to above, the transducer 102 is detachably mounted to the module 103. As described more fully herein, by providing for selective attachment and detachment of the transducer 102, a medical practitioner is accorded the ability to select a different transducer type based on the particular test/measurement undertaken; and without having to select and stock an entirely different assembly. As should be appreciated, this option beneficially allows the medical facility to reduce its capital expenditure by stocking one module for multiple types of transducers, rather than having to stock a complete ultrasound assembly for each type of transducer.

Similarly, the display 104 is illustratively detachably mounted to module 103. As mentioned above, the display 104 may be detached for optimal placement in the field of view of the sonographer during the imaging session. The data connection between the display 104 and the module 103 may be wired, as with USB or similar high-speed serial interface, or wireless, as with an ultra-wideband (UWB) protocol promoted by the WiMedia Alliance. If wireless, the display 104 should include a provision to provide power, such as a battery or DC input connector for an AC adapter.

Taking advantage of the detachable feature of the display 104, the medical facility is afforded the ability to reduce their overall capital investment or increase their aggregate ultrasound scanner reliability, or “up time”, by separately stocking detachable display units. The display units may then be combined at will with one or more ultrasound transducers and modules as the patient workload changes, or as display units occasionally fail.

In representative embodiments, the transducer 102 is magnetically connected to the module 103. Alternatively, the transducer 102 may be mechanically connected to the module 103, such as by latching mechanisms (not shown) or friction-fit (i.e., ‘snap-on’) mechanisms. As described more fully below, the transducer 102 is connected electrically to the module by an interface (not shown in FIG. 1), which is operative to provide electrical power to the transducer 102 and to pass electrical signals from the transducer 102. Illustratively, the electrical-mechanical connection may comprise tabs (not shown) comprising copper with gold coating on a lower end (not shown) of the transducer 102 that mate to the electrical tabs (not shown) on the module 103 end. A skirt may be located around either the transducer 102 or module 103 sides that align the module to the opposite end. The connected structure is sealed such that it is resistant to fluid ingress. For example, the electrical-mechanical connection of the transducer 102 to the module 103 can be made similarly as described in U.S. Pat. No. 6,635,019, the disclosure of which is specifically incorporated herein by reference.

Notably however, and as described below, because of the microbeamformer placed in the transducer 102, the electrical connections needed to mate are much reduced since less analogue signals are required thus allowing for simpler mechanical connections such as “snap-on” mechanism where the mechanical tolerance required is much less. The is much more practically achievable allowing for easy connect/disconnect modules where the wear and tear over time would still allow a robust electrical connection.

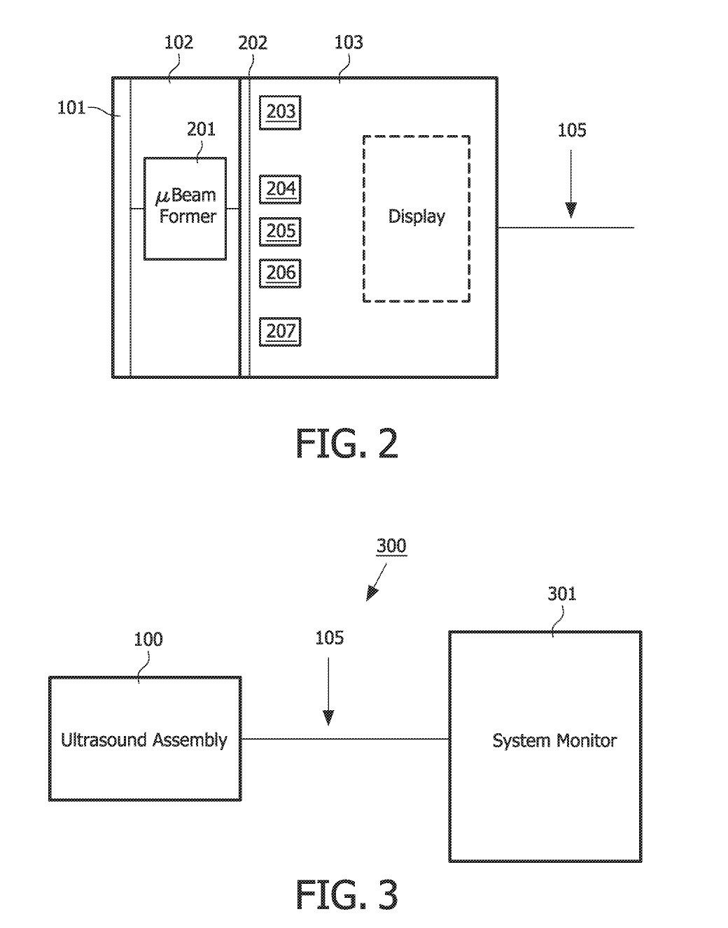

FIG. 2 is a simplified schematic diagram of an ultrasound assembly 200 in accordance with a representative embodiment. The assembly 200 includes many common features to the assembly 100 described in connection with FIG. 1. Such common features are often not duplicatively described, but may be further elaborated upon.

The transducer 102 comprises transducer elements 102 as noted above. The transducer elements 101 may be linear array or a phased array, or a combination thereof, such as described in U.S. Pat. No. 6,436,048. The beam from the transducer elements 101 may also be steered as described in U.S. Pat. No. 7,037,264. As noted, the transducer elements 101 may be a curved linear (1D) array (CLA), such as described in U.S. Pat. No. 6,540,682. These patents are assigned to the present assignee and are all specifically incorporated herein by reference.

The transducer 102 also comprises a microbeamformer 201. The microbeamformer 201 may be as described in U.S. Pat. No. 6,436,048. Echoes by the elements 101 of the transducer 102 are partially beamformed by a micro-beamformer 201. In a representative embodiment, the micro-beamformer 201 contains circuitry which controls the signals applied to groups of elements (“patches”) of the transducer elements 101 and effects some processing of the echo signals received by elements of each group. Micro-beamforming in the transducer 102 beneficially reduces the number of conductors in the connection 105 between the assembly 100 and the ultrasound system (not shown). Additional details of the benefits derived from microbeamforming may be found in commonly assigned U.S. Pat. No. 5,997,479, the disclosure of which is specifically incorporated herein by reference and in the '048 patent.

In addition to the benefits derived from dividing the beamforming with a microbeamformer, the representative embodiments foster additional benefits because the microbeamformer 201 is co-located with the transducer elements 101 within the transducer 102. For example, superior electrical performance is realized because the electronics of the microbeamformer 201 are proximal to the elements 101, eliminating the need for complex interconnects, cabling, and the attendant signal distortions and power losses of long electrical connections.

Moreover, microbeamforming may be specifically matched with the type of array of transducer elements 101, since the microbeamforming is physically combined with the elements 101. Moreover, because of the matching of the microbeamformer 201 to the particular type of sensor array different versions of the microbeamformer 201 can be optimized for different sensor classes (e.g., sector, linear, CLA) and for different frequencies/impedances. Thus, rather than a generic microbeamformer that is configured to work acceptably with each of a number of transducer types, the present teachings allow for an improved if not optimal match of microbeamformer to the type of transducer array of each individual transducer 102.

Illustratively, the microbeamformer 201 may be matched in dimensions to the layout of the acoustic elements of sensor array 101 and then may be mounted directly to the sensor itself, saving space, simplifying the interconnection scheme between the microbeamformer 201 and the sensor, and reducing electrical noise and signal loss by minimizing signal trace lengths.

In addition, the microbeamformer 201 may be optimized to respond to the resonant frequency range of the acoustic sensor elements and to apply beamforming delays that match said frequency range with sufficient resolution for high quality imaging, but not so much resolution as to waste circuit components. Similarly, the microbeamformer circuitry may be optimized to match the characteristic impedance of the sensor elements 101.

As described above, the transducer 102 is connected to the module 103 via an interface 202; and the interface 202 comprises both a mechanical connection and an electrical connection. The mechanical connection enables attaching and detaching of the transducer 102 to the module 103 as described above. The electrical connection provides power to the transducer 102, in particular to its integrated microbeamformer; and signals from the microbeamformer 201 to the module 103 for further processing. The electrical mechanical connection can be made using a standard USB type latching connector or a custom mechanical latch type, snap fit, or magnetic type connection, such as described in co-pending U.S. Patent Application Ser. No. 60/941,427 entitled Wireless Ultrasound Probe Cable and filed on Jun. 1, 2007. The disclosure of this application is specifically incorporated herein by reference.

The module 103 comprises a scan controller 203 and a main beamformer 204, such as described in U.S. Pat. No. 6,436,048 or in U.S. Pat. No. 7,037,264 for example. The module 103 may also comprise DSP circuitry 205 for the signal detection path in multiple modes (e.g., Greyscale, Flow, PW, CW). In addition, the module 103 comprises a power supply 206 for powering the module 103, the transducer 102 and the display component 104. It also comprises a memory 207 for storing acquired images user presets scan control and beamforming coefficients user programs.

The power supply 206 may be an AC/DC converter operative to provide a desired DC voltage. Alternatively, the power supply 206 may be a known type of battery. The implementation of the latter provides certain benefits over known devices. First, because no cable is needed for power, the assembly 100 may be readily implemented according to a wireless protocol providing ease of portability and use. Moreover, a battery, which can be rechargeable, can be recharged simultaneously with data transfer over the same (wired) connection 105. For example, a USB connection may be used to realize both data and power for recharging.

The use of a battery also accords the benefit of powering the display 104 in a local manner. Thus, the display 104 does not require a remote power supply, and may have its own battery. As such, the display 104 can be compact and light. By contrast, a separate monitor or external display such as on a personal digital assistant (PDA) will require a power source and central processing unit (CPU), which add to the complexity of the system and reduce the ergonomic benefits derived from the self-contained assembly 100.

Furthermore, the rendering and formatting of images may be effected in the module 103, thus minimizing the need for processing at the display 104. This reduces not only the size and weight of the system, but also the cost of the display 104. The display 104 is easily connected or disconnected to the module 103 thus allowing for flexibility in positioning for the user. Due to the few electrical signals required, the mechanical-electrical connection can be made to be simple since the alignment and tolerance of the electrical tabs easily achievable. The electrical tabs of the module (described above) can mate to the electrical tabs of the display 104, such as by a magnetic connection, a friction fit or some other type of latching connection. This mechanical connection can allow for rotation and tilting of the display.

FIG. 3 is a simplified block diagram of a system 300 for ultrasound imaging in accordance with a representative embodiment. The system 300 comprises the assembly 100 and a system monitor 301 connected by connection 105 as shown. The system 300 includes many common features and details to those described in connection with the representative embodiments of FIGS. 1 and 2.

The system monitor 301 may be a stand-alone monitor used by the medical practitioner using the assembly 100 and may be in lieu of or in addition to the display 104. Alternatively, the system monitor 300 may be a central unit (e.g., a server) of a medical facility that provides access to the images from the assembly in real-time or via memory. Again, the link between the assembly 100 and the monitor 301 may be wired or wireless, as may the link from the monitor to other devices of a network connected thereto.

In view of this disclosure it is noted that the various ultrasound assemblies and systems ultrasound imaging may comprise a variety of devices, components, software, hardware and firmware. Moreover, applications other than medical imaging may benefit from the present teachings. Further, the various devices, components, software, hardware, firmware and parameters are included by way of example only and not in any limiting sense. In view of this disclosure, those skilled in the art can implement the present teachings in determining their own applications and needed devices, components, software, hardware and firmware to implement these applications, while remaining within the scope of the appended claims.

1. An ultrasound assembly, comprising:
a module having an input side and an output side;
a ultrasound transducer comprising a micro-beamformer configured for attachment and detachment from the input side of the module; and
a display attached to the output side of the module.

2. An ultrasound assembly as claimed in claim 1, wherein the ultrasound transducer comprises a linear transducer array and the module is configured to receive input signals from the linear transducer array and to provide output signals to the display.

3. An ultrasound assembly as claimed in claim 1, wherein the ultrasound transducer comprises a phased array transducer array and the module is configured to receive input signals from the phased array transducer array and to provide output signals to the display.

4. An ultrasound assembly as claimed in claim 1, wherein the ultrasound transducer comprises a curved transducer array and the module is configured to receive input signals from the curved transducer array and to provide output signals to the display.

5. An ultrasound assembly as claimed in claim 1, wherein the module comprises a microcontroller and a memory and the microcontroller is configured to acquire a transducer parameter from the memory.

6. An ultrasound assembly as claimed in claim 5, wherein the microcontroller is configured to receive data from the transducer array after acquiring the transducer parameter.

7. An ultrasound assembly as claimed in claim 5, wherein the microcontroller is configured to optimize calculating configuration and scanning coefficients of the ultrasound transducer.

8. An ultrasound assembly as claimed in claim 1, wherein the display is disposed over the module.

9. An ultrasound assembly as claimed in claim 1, wherein the module and the transducer are configured to mechanically attach and detach from one another.

10. An ultrasound assembly as claimed in claim 1, wherein the display is electrically connected to the assembly in a wired manner.

11. An ultrasound assembly as claimed in claim 1, wherein the module and the display are configured to magnetically attach and detach from one another.

12. An ultrasound assembly as claimed in claim 1, wherein the display is electrically connected to the assembly in a wireless manner.

13. A system for ultrasound imaging, comprising:
an ultrasound assembly, comprising: a module having an input side and an output side; an ultrasound transducer comprising a micro-beamformer configured for attachment and detachment from the input side of the module; and a display attached to the output side of the module.

14. A system as claimed in claim 13, wherein the ultrasound transducer comprises a linear transducer array and the module is configured to receive input signals from the linear transducer array and to provide output signals to the display.

15. A system as claimed in claim 13, wherein the ultrasound transducer comprises a phased array transducer array and the module is configured to receive input signals from the phased array transducer array and to provide output signals to the display.

16. A system as claimed in claim 13, wherein the ultrasound transducer comprises a curved transducer array and the module is configured to receive input signals from the curved transducer array and to provide output signals to the display.

17. A system as claimed in claim 13, wherein the ultrasound transducer comprises a memory and the module comprises a microcontroller configured to acquire a transducer parameter from the memory.

18. A system as claimed in claim 17, wherein the microcontroller is configured to receive data from the transducer array after acquiring the transducer parameter.

19. A system as claimed in claim 13, wherein the microcontroller is configured to optimize calculating configuration and scanning coefficients of the ultrasound transducer.

20. A system as claimed in claim 13, wherein the display is disposed over the module.

21. A system as claimed in claim 13, wherein the module and the transducer are configured to mechanically attach and detach from one another.

23. A system as claimed in claim 13, wherein the mechanical attachment is by friction-fit.

23. A system as claimed in claim 13, further comprising another display remote to the ultrasound assembly.
